(19) United States
(12) Patent Application Publication
(10) Pub. No.: US 2020/0010347 A1
(43) Pub. Date: Jan. 9, 2020

(54) PROCESS FOR REMOVAL AND RECOVERY OF COPPER-CYANIDE COMPLEX USING MICROALGAE

(71) Applicant: Symbiosis International Deemed University, Maharashtra (IN)

(72) Inventor: Yogesh PATIL, Maharashtra (IN)

(21) Appl. No.: 16/490,688

(22) PCT Filed: Mar. 3, 2018
(86) PCT No.: PCT/IB2018/051365

§ 371 Date: Sep. 3, 2019

(30) Foreign Application Priority Data
Mar. 3, 2017 (IN) 201721007663

(51) Int. Cl. C02F 3/32; C12N 1/12; C02F 1/28

(52) U.S. Cl. CPC C02F 3/322; C02F 2101/18; C02F 1/286; C12N 1/12

(57) ABSTRACT

A process of removal and recovery of metal-cyanide complex from effluents is disclosed. The process includes isolating a species of Scenedesmus via enrichment culture technique in alkaline medium. The process further includes releasing the species of Scenedesmus, after being isolated, in the effluents at predetermined conditions including a predefined pH and a predefined temperature. The species of Scenedesmus released in the effluents at the predetermined conditions: degrades cyanide moiety of a metal-cyanide present in the effluents and releases the metal ions; utilizes the carbon and nitrogen from the cyanide or metal cyanide complex present in the effluents; accumulates a first fraction of metal ions from the solution; and bio-sorbs the second fraction of the metal ions onto the cells of the microalgae thereby resulting in the removal of both the cyanide and the metal from the effluents. The metal-cyanide complexes comprise at least one of a copper-cyanide and a zinc-cyanide.

### TECHNICAL FIELD

The present invention in general relates to a field of treatment of an industrial waste. More particularly, the invention relates to a process for removal and recovery of copper-cyanide complex from the industrial waste using microalgae.

### BACKGROUND

In the recent times, due to enormous industrial and commercial growth, the world has witnessed tremendous increase in the number of various industries being deployed for manufacturing/production of different products. The manufacturing of products requires adopting various kinds of materials which are then discharged as effluents in the surrounding environment. In order to prevent damages to the human as-well-as flora and fauna of the surrounding environment, the industrial waste has to be treated before being discharged in the environment. Therefore, more effective and efficient industrial waste management is desired. This is because, if the industrial waste is managed haphazardly, the environmental pollution caused due to hazardous and toxic waste waters emanated from diverse industries may result in creating a severe impact on all classes of life.

Some of the toxic chemicals that are emanated from industrial processes include cyanide and/or metal complexes (Cu, Zn, etc.) of cyanide. The cyanide and/or the metal complexes of cyanide may inhibit sensitive enzyme cytochrome oxidase in living cells. Though the cyanide is toxic in nature, however, the cyanide could be utilized in large applications of several industrial processes including, but not limited to, mining, electroplating, manufacturing of printed circuit boards, jewelry units and automobiles. These industries may further consequently generate large quantities of free cyanide and metal-cyanide bearing effluents.

Due to its hazardous and toxic nature, the cyanide has been listed as a priority environmental pollutant by the USA agency, US EPA. In addition to this, various other statutory agencies across the world have been adopting strict regulations for complete removal of the cyanide and the metal-cyanide from the industrial waste prior to discharging of the industrial waste or recycling thereof.

In the existing art, several physical and/or chemical processes have been proposed to manage cyanide containing effluents. However, these existing processes are suffering from the problems such as cost effectiveness, efficacy and requirement of special equipment and maintenance thereof. Thus, there is a need of developing an alternative to make a paradigm shift in the waste treatment area with simultaneous adaptation to climate change.

In the recent past, use of biological technologies has been seen as an effective alternative to the physical and/or chemical processes because of several advantages. However, most biological methods make use of either bacterial or fungal organisms, which require nutrients and emanate carbon-dioxide during treatment process resulting to Greenhouse (GHG) emissions. Thus, there exists a long-felt need for exploring an alternative sustainable strategy to manage the cyanide containing effluents for environmental clean-up and safety.

### SUMMARY

Before the present processes and related apparatuses/components for implementing the processes are described, it is to be understood that this disclosure is not limited to the particular processes or methods, apparatuses and their arrangement as described, as there can be multiple possible embodiments which are not expressly illustrated in the present disclosure but may still be practicable within the scope of the invention. It is also to be understood that the terminology used in the description is for the purpose of describing the particular versions or embodiments only and is not intended to limit the scope of the present application. This summary is not intended to identify essential features of the subject matter nor it is intended for use in detecting or limiting the scope of the subject matter.

In accordance with embodiments of the present disclosure, a process of removal and recovery of metal-cyanide complex from effluents is disclosed. The process may include isolating a species of Scenedesmus via enrichment culture technique in an alkaline medium. The process may further include releasing the species of Scenedesmus, after being isolated, in the effluents at predetermined conditions including a predefined pH and a predefined temperature. The species of Scenedesmus released in the effluents at the predetermined conditions may degrade cyanide moiety of a metal-cyanide present in the effluents and releases the metal ions. The species of Scenedesmus released in the effluents at the predetermined conditions may utilize the carbon and nitrogen from the cyanide or metal cyanide complex present in the effluents. The species of Scenedesmus released in the effluents at the predetermined conditions may accumulate a first fraction of metal ions from the solution. The species of Scenedesmus released in the effluents at the predetermined conditions may further bio-sorb the second fraction of the metal ions onto the cells of the microalgae thereby resulting in the removal of both cyanide and copper from the effluent.

### BRIEF DESCRIPTION OF DRAWINGS

The detailed description is described with reference to the accompanying FIGURES. In the FIGURES, the left-most digit(s) of a reference number identifies the FIGURE in which the reference number first appears. The same numbers are used throughout the drawings to refer like features and components.

FIG. 1 illustrates a process of removal and recovery of metal-cyanide complex using microalgae, in accordance with an embodiment of the present subject matter.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items.

It must also be noted that, the singular forms “a,” “an,” and “the” include plural references unless the context clearly dictates otherwise. Although any methods similar or equivalent to those described herein can be used in the practice or testing of embodiments of the present disclosure, the exemplary methods are now described. The disclosed embodiments are merely exemplary of the disclosure, which may be embodied in various forms.

Various modifications to the embodiment will be readily apparent to those skilled in the art and the generic principles herein may be applied to other embodiments. However, one of ordinary skill in the art will readily recognize that the present disclosure is not intended to be limited to the embodiments illustrated but is to be accorded the widest scope consistent with the principles and features described herein.

The present disclosure describes a process for removal and recovery of metal-cyanide complex using microalgae. The microalgae being photosynthetic in nature, has a potential of utilizing fewer nutrients, an advantage of energy conservation and mitigation of global warming by way of absorbing carbon dioxide. The metal in the metal complex may be at least one of copper (Cu), zinc (Zn), and the like. The microalgae may include Scenedesmus species.

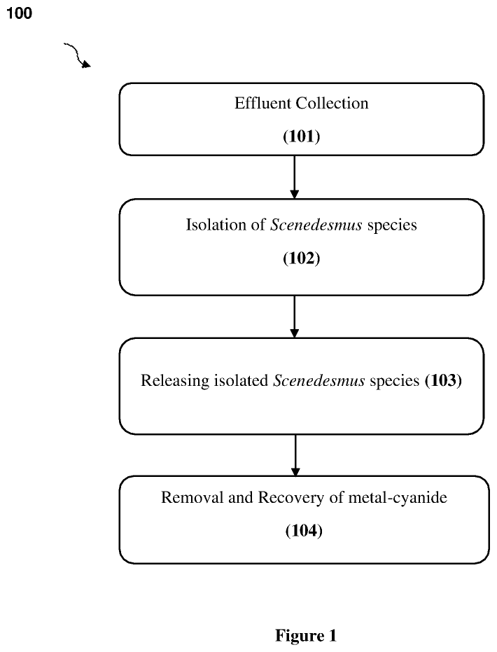

Now referring FIG. 1, a process of removal and recovery of metal-cyanide complex using microalgae is illustrated, in accordance with an embodiment of the present subject matter.

As shown in FIG. 1, at step 101, effluents containing metal-cyanides comprising copper cyanides from the industries is transferred to canals or to large tanks where the effluents may be treated before releasing them in aquatic bodies. The collection of the effluents may be in-house or at a place (e.g. a warehouse) away from the industry. The warehouse may accept the effluents from all the industries and may further combine the effluents to get a greater amount of metal-cyanides comprising copper cyanides.

At step 102, species of Scenedesmus may be isolated via an enrichment culture technique. In one embodiment, the species of Scenedesmus may be Scenedesmus bijugatus. In one embodiment, a process of treatment of free cyanide and copper-cyanide (tetracyanocuprate i.e. TCC) may be carried out using the Scenedesmus species of the microalgae. The species Scenedesmus may be isolated via the enrichment culture technique in an alkaline condition. The enrichment culture is an artificial medium with specific and known qualities (e.g. containing cyanide) that favors the growth of only desired type of microorganisms while inhibiting the growth of others. The microbial source required for the enrichment culture may be obtained from different segments of environment (e.g. soil sample, water sample and the like). Thus, the Scenedesmus species capable of degrading the cyanide and/or metal-cyanide may be isolated using the enrichment culture technique.

At step 103, the Scenedesmus species of the microalgae may be further released into the effluents. In one embodiment, the effluents may be industrial wastes. The Scenedesmus species of the microalgae may remove the cyanide from the effluent and recover the metal from metal-cyanide complex of the solution. The species of Scenedesmus may degrade cyanide moiety of metal-cyanide and may further release the metal ions into the effluents. The species of Scenedesmus may utilize the carbon and nitrogen from the cyanide or a metal cyanide complex present in the effluents. The species of Scenedesmus may accumulate a first fraction of metal ions from the solution. The species of Scenedesmus may bio-sorb a second fraction of the metal ions onto the cells of the microalgae thereby resulting in the removal of both the cyanide and the metal from the effluents.

In one embodiment, the aforementioned process at step 103 of removing cyanide and recovering metal from metal-cyanide complex may be carried out at predetermined conditions including, but not limited to, pH, temperature, cell density and the like. In one embodiment, different combinations of each of these conditions may be tested in order to obtain a suitable combination that yields best removal/recovery of the cyanide and the metal from the metal-cyanide complex of the effluent. In one embodiment, the process may be optimized for a pH value within a predefined range of 4 to 10. In another embodiment, the process may be optimized by maintaining a temperature within a predefined range of 25° C. to 45° C.

In accordance with embodiments of the present disclosure, the metal-cyanide complexes include at least one of a copper-cyanide and a zinc-cyanide. In one exemplary embodiment, the metal-cyanide complexes include a copper-cyanide. In this exemplary embodiment, the species Scenedesmus may degrade the cyanide moiety of copper-cyanide (TCC) and may further release the copper ions into the effluents. In this exemplary embodiment, the cells of the microalgae may enable both sorption and accumulation of the copper ions. In this exemplary embodiment, the copper ions may fractionally get bound onto cells of the microalgae while the other fraction of the copper ions may get accumulated in microalgae biomass.

It must be noted herein that since the microalgae has an ability to undergo photosynthesis process (like any other plant), the microalgae may utilize inorganic carbon substrate. The photosynthesis ability of the microalgae may make the microalgae suitable for carbon dioxide (CO₂) mitigation. The microalgae may consume essential nutrients (such as carbon and nitrogen) of the cyanide and/or metal-cyanide complex present in the solution.

In the aforementioned exemplary embodiment of biodegradation of the TCC, the species of Scenedesmus may accumulate approximately 65% of the total copper ions removed from the solution and may further bio-sorb approximately 35% of the copper ions onto cells of the microalgae. Thus, both cyanide and copper may be removed/recovered from the solution.

At step 104, the cells of the microalgae (alive or dead) having the metal ions may be further transferred into an aqueous solution facilitating separation of the metal from the cells of the microalgae. For example, in the aforementioned exemplary embodiment of biodegradation of the TCC, the cells of the microalgae (alive or dead) having the copper ions may be further transferred into the aqueous solution wherein the copper may be separated from the cells of the microalgae

It must be noted herein that, in certain cases, heavy metals may be precious and may have finite resources. Therefore, the heavy metals may be recovered using such microorganisms (alive or dead) in suitable species form.

In accordance with embodiments of the present disclosure, the process for removal and recovery of copper-cyanide complex using microalgae described above may have following advantages:
Being photosynthetic in nature, microalgae has the potential of utilizing fewer nutrients, an advantage of energy conservation and mitigation of global warming/climate change by way of absorbing carbon dioxide.
Recovery of metal is possible in suitable species form.
Complete removal of metal-cyanides from the effluents is possible since the microalgae culture not only accumulates the metal inside the cells but further adheres the metal on its cell wall (i.e. using biosorption).
Complete removal of metal-cyanides from the effluents is achieved due to predominant biological reaction and minimal or no auto-oxidation (non-biological reaction) of cyanide.

The embodiments, examples and alternatives of the preceding paragraphs, the description, including any of their various aspects or respective individual features, may be taken independently or in any combination. Features described in connection with one embodiment are applicable to all embodiments, unless such features are incompatible.

The present disclosure can be embodied in many other forms or carried out in other ways, without departing from the spirit or essential characteristics thereof, and the above-mentioned embodiment of the disclosure have been disclosed in detail only for illustrative purposes. It is understood that the disclosure is not limited thereto but is susceptible of numerous changes and modifications as known to those skilled in the art, and all such variations or modifications of the disclosed system, including the rearrangement of parts, lie within the scope of the present disclosure.

The foregoing description shall be interpreted as illustrative and not in any limiting sense. A person of ordinary skill in the art would understand that certain modifications could come within the scope of this disclosure.

Although implementations for have been described in language specific to features and/or processes, it is to be understood that the disclosure is not necessarily limited to the specific features or processes described. Rather, the specific features and processes are disclosed as examples of implementations for removal and recovery of copper-cyanide complex using microalgae.

1. A process of removal and recovery of metal-cyanide complex from effluents, comprising: isolating a species of Scenedesmus microalgae via an enrichment culture technique; and
releasing the species of Scenedesmus, after being isolated, in the effluents at predetermined conditions including a predefined pH and a predefined temperature, wherein the species of Scenedesmus released in the effluents at the predetermined conditions
degrades cyanide moiety of a metal-cyanide present in the effluents and releases the metal ions,
utilizes the carbon and nitrogen from the cyanide or a metal cyanide complex present in the effluents,
accumulates a first fraction of metal ions from the solution, and
bio-sorbs a second fraction of the metal ions onto the cells of the microalgae thereby resulting in the removal of both the cyanide and the metal from the effluents.

2. The process according to claim 1, wherein the metal-cyanide complexes comprise at least one of a copper-cyanide and a zinc-cyanide.

3. The process according to claim 1, wherein the isolation of Scenedesmus species via the enrichment culture technique is carried in an alkaline condition.

4. The process according to claim 1, wherein the cells of the Scenedesmus species of microalgae exhibits both the accumulation and the sorption of the copper ions.

5. The process according to claim 1, wherein the predefined pH is in a range of 4 to 10.

6. The process according to claim 1, wherein the predefined temperature is in a range of 25° C. to 45° C.

7. The process according to claim 1, wherein the accumulation of the first fraction of the metal ions from the solution ranges from 60% to 70%.

8. The process according to claim 1, wherein the accumulation of the second fraction of metal ions from the solution ranges from 30% to 40%.

9. The process according to claim 1, wherein the species Scenedesmus is Scenedesmus bijugatus.

10. The process according to claim 1, wherein the cells of the microalgae having the metal ions are further transferred into an aqueous solution facilitating separation of the metal from the cells of the microalgae.
